(19) United States
(12) Patent Application Publication
(10) Pub. No.: US 2005/0025723 A1
(43) Pub. Date: Feb. 3, 2005

(54) Sunburn treatment and sunburn prevention method

(76) Inventor: James M. Lipton, Woodland Hills, CA (US)

Correspondence Address: PERKINS COIE LLP, POST OFFICE BOX 1208, SEATTLE, WA, 98111-1208, US

(21) Appl. No.: 10/853,902

(22) Filed: May 25, 2004

Related U.S. Application Data
(63) Continuation of application No. 09/704,327, filed on Nov. 1, 2000, now abandoned.

(51) Int. Cl. A61K 7/42; A61K 38/08; A61K 38/05

(52) U.S. Cl. 424/59; 514/16; 514/17; 514/18

(57) ABSTRACT

The present invention is directed to a treatment for sunburn and a method for preventing sunburn. One aspect of this invention involves a sunburn treatment comprising one or more polypeptides with an amino acid sequence including KPV (SEQ. ID. NO. 1), MEHFRWG (SEQ. ID. NO. 2), HFRWGKPV (SEQ. ID. NO. 3), or SYSMEHFRWGKPV (SEQ. ID. NO. 4) for the treatment of the cutaneous inflammation caused by exposure to ultraviolet radiation. The polypeptides are at a level to effectively treat the cutaneous inflammation and are carried by a carrier. The one or more polypeptides can also be a dimer formed from any of the amino acid sequence above. In one preferred embodiment of the invention, the one or more polypeptides are used to prevent sunburn. In another preferred embodiment, the one or more polypeptides are dissolved in a carrier. In another preferred embodiment, the carrier includes aloe vera and lidocaine hydrochloride. In another preferred embodiment of the invention, the one or more polypeptides are dissolved in a liquid that is associated with an absorbent material for application to sunburned skin.

### FIELD OF THE INVENTION

The present invention relates to the field of preventing and treating sunburns.

### BACKGROUND OF THE INVENTION

The skin is one of the largest body organs and functions as one of its major interfaces with the environment, including solar radiation. Exposure to solar radiation has the beneficial effects of stimulating the cutaneous synthesis of vitamin D and providing radiant warmth. Foster, J., Sunburn, eMedicine—Online Medical Reference Textbook, (last modified May 1, 2000), <http://emedicine.com/emerg/topic798.htm>. Unfortunately, when the skin is subjected to excessive radiation in the ultraviolet range, deleterious effects, such as sunburn, occur. Id. Sunburn is an acute cutaneous inflammatory reaction that follows excessive exposure of the skin to ultraviolet radiation (UVR). The inflammatory response occurs within 2-6 hours after exposure and peaks at 20-24 hours with symptoms such as erythema, warmth, tenderness, edema, and blistering (severe cases). Id.

Solar radiation is categorized along the electromagnetic spectrum that is divided according to wavelength into ultraviolet (less than 400 nm), visible (400-760 nm) and infrared (greater than 760 nm). Bickers, D., Sun-Induced Disorders, Emergency Medicine Clinics of North America, 3(4): 659-663, 660 (1985). Ultraviolet radiation has been arbitrarily divided into three major components, the UVA (320 to 400 nm); the UVB (290 to 320 nm); and the UVC (200 to 290 nm). The UVC is completely absorbed by stratospheric ozone and does not reach the earth's surface. Id. UVB radiation is 1000 times more potent at inducing solar erythema than UVA and is, therefore, the principle cause of sunburn. Id. at 661. UVA comprises the majority of ultraviolet radiation that reaches the surface of the earth (about 90% at midday) and therefore accounts for a significant percentage of the acute and chronic cutaneous effects of UVR. Foster, supra. Further, since the advent of tanning salons utilizing ultraviolet wavelengths in the UVA-spectrum, UVA-induced sunburn is becoming more common place. Cavello, J. and Deleo, V., Sunburn Dermatologic Clinics, 4(2): 181-187, 181 (1986).

The minimal single dose of UVR required to produce erythema at an exposed site is known as the minimal erythema dose (MED). Moderate to severe sunburn occurs at 3-8 MEDs. Foster, supra. Irradiance, the rate of radiation delivery at the skin surface, and the duration of the irradiation also effect the sunburn response. The same magnitude of response is achieved by doubling the irradiance and halving the irradiation period. This reciprocal relationship between intensity and time holds true for the delayed erythema response to UVR over a very wide range of times and irradiances up to 11 orders of magnitude. Fitzpatrick, et al., Acute Effects of Ultraviolet Radiation on the Skin: The Sunburn Reaction, Dermatology in General Medicine, 4^{th} Edition, 1651-1655, 1651 (1993).

The precise biochemical pathways that lead to the sunburn reaction are not well understood, but appear to involve multiple inflammatory mediators, including histamine, prostaglandins and cytokines. Id. Histamine levels are elevated in suction blister fluid obtained during UVB- and UVA-induced erythema in humans, returning to normal after 24 hours although erythema is still prominent. Prostaglandin E₂ increased to approximately 150 percent of control levels after 24 hours and then diminished. Prostaglandins are known to evoke redness and pain in human skin after intradermal injection, and their presence in elevated amounts in suction blisters after UV irradiation suggests their involvement in sunburn erythema. Id. at 1654. UV irradiation is a potent stimulus for keratinocyte release of cytokines with local and systemic proinflammatory and immunomodulatory actions. Id.

Erythema induced by UVA also is associated with histologic evidence of epidermal injury but this is much less pronounced than that which occurs after UVB. In the dermis, polymorphonuclear leukocytes and lymphocytes are seen in increased numbers. Endothelial cell enlargement also occurs. Bickers, supra, at 662.

Severity of sunburn is related to duration of exposure, skin type and amount of protection. Potts, Sunlight, Sunburn, and Sunscreens, Postgrad. Med., 87:52-61 (1990). Factors influencing the cutaneous response to UVR depend on interactions among many other factors besides exposure time and dose. Wavelengths of the radiation source, skin characteristics such as pigmentation, hydration and skin thickness, and external factors such as wind, temperature and humidity all effect the response. Fitzpatrick, supra at 1652. Reflection off snow and sand may also lead to increased exposure. Foster, supra. Some medications are known to be sensitizing to ultraviolet radiation. Tricyclic antidepressants, antihistamines, anti-infectives, antineoplastic agents, antipsychotic agents, diuretics, hypoglycemic agents, nonsteroidal anti-inflammatory drugs, and sunscreens all may decrease an individual's tolerance for sun exposure. Potts, supra at 54.

Current treatment for sunburn includes the systemic administration of aspirin or nonsteroidal anti-inflammatory drugs (NSAIDs) to inhibit the cyclooxygenase pathway and thereby reduce prostaglandin production. NSAIDs work best if administered within the first several hours after exposure. Bickers, supra at 663. Systemic corticosteroids are often employed and probably shorten the course of the pain that accompanies severe sunburn. Id. Corticosteroids should not be given to patients with severe, second-degree burns because they increase the risk of infection. Foster, supra. Topical steroids show minimal, if any benefit. Id.

Over-the-counter topical remedies include anesthetics such as lidocaine hydrochloride, benzocaine, and pramoxine hydrochloride. Skin soothing ingredients such as aloe vera, tocopheryl acetate (Vitamin E), menthol, camphor, eucalyptus oil, and calamine are also popular ingredients known in the art.

Home topical remedies include taking a cool bath with oatmeal or baking soda; and spreading the juice of a cut potato, lavender essential oil, or chamomile on the burn. Mayhall, Ten Home Remedies for Sunburn, Seasonal Health, (Jul. 14, 2000), <http://drkoop.com/wellness/seasonal/summer/sunburn.html>.

While these topical remedies may help soothe the skin or temporarily relieve the pain associated with sunburn, they are not a treatment for the underlying inflammation that defines sunburn. Non-steroidal anti-inflammatories must be given systemically soon after exposure to be effective. Moreover, patients with allergies to NSAIDs, sensitive stomachs, or potential negative drug interactions may not be able to tolerate this treatment. Thus, a topical treatment that effectively reduces sunburn inflammation is needed.

Current sunburn prevention methods include wearing protective clothing and avoidance of the sun during midday. But these methods restrict the outdoor activities, such as swimming, of a person wanting to avoid sunburn.

Topical products for the prevention of sunburn fall into two categories: physical barriers and chemical absorbers. Chemical sunscreens are generally aromatic compounds conjugated with a carbonyl group. After application, the chemical sunscreen components diffuse into the stratum corneum and adsorb or conjugate with various proteins. Product effectiveness is determined by the depth of penetration, binding affinity for different proteins, and duration of protection. These chemicals absorb radiation in the UV spectrum. Potts, supra at 54. Chemical sunscreens have the disadvantages of possibly staining clothing and causing contact dermatitis. Id. Moreover, recently concerns have been raised regarding the mutagenic properties of the most popular chemical sunscreens p-amino-benzoic acid (PABA) and PABA esters. Id.

Physical blockers, such as zinc oxide, talc, and titanium dioxide, reflect or scatter UVR. Many consumers find these products messy to apply and cosmetically unappealing. Id.

Thus, a sunburn prevention method that provides another option to avoid the painful inflammation of sunburn is needed.

### SUMMARY OF THE INVENTION

The present invention is directed to a treatment for sunburn and a method for preventing sunburn. One aspect of this invention involves a sunburn treatment comprising one or more polypeptides with an amino acid sequence including KPV (SEQ. ID. NO. 1), MEHFRWG (SEQ. ID. NO. 2), HFRWGKPV (SEQ. ID. NO. 3), or SYSMEHFRWGKPV (SEQ. ID. NO. 4) for the treatment of the cutaneous inflammation caused by exposure to ultraviolet radiation. The polypeptides are at a level to effectively treat the cutaneous inflammation and are carried by a carrier. The one or more polypeptides can also be a dimer formed from any of the amino acid sequence above. In one preferred embodiment of the invention, the one or more polypeptides are used to prevent sunburn. In another preferred embodiment, the one or more polypeptides are dissolved in a carrier. In another preferred embodiment, the carrier includes aloe vera and lidocaine hydrochloride and is used to treat sunburn. In another preferred embodiment of the invention, the one or more polypeptides are dissolved in a liquid that is associated with an absorbent material for application to sunburned skin.

### BRIEF DESCRIPTION OF THE DRAWINGS

FIG. 1 shows a representation of the chemical structure of one form of the KPV dimer for use with one aspect of the invention.

### GENERAL DESCRIPTION OF THE INVENTION

The references cited below are hereby incorporated by reference as if fully set forth herein. The present invention involves a treatment, methods of treatment and prevention, and a treatment packet for sunburn with the use of alpha-melanocyte stimulating hormone (“α-MSH”) and/or its derivatives. α-MSH is an ancient thirteen amino-acid peptide (SEQ. ID. NO. 4) produced by post-translational processing of the larger precursor molecule propiomelanocortin. It shares the 1-13 amino acid sequence with adrenocorticotropic hormone (“ACTH”), also derived from propiomelanocortin. α-MSH is known to be secreted by many cell types including pituitary cells, monocytes, melanocytes, and keratinocytes. It can be found in the skin of rats, in the human epidermis, or in the mucosal barrier of the gastrointestinal tract in intact and hypophysectomized rats. See e.g. Eberlie, A. N., The Melanotrophins, Karger, Basel, Switzerland (1998); Lipton, J. M., et. al., Anti-inflammatory Influence of the Neuroimmunomodulator α-MSH, Immunol. Today 18, 140-145 (1997); Thody, A. J., et. al., MSH Peptides are Present in Mammalian Skin, Peptides 4, 813-815 (1983); Fox, J. A., et. al., Immunoreactive α-Melanocyte Stimulating Hormone. Its Distribution in the Gastrointestinal Tract of Intact and Hypophysectomized Rats, Life. Sci. 18, 2127-2132 (1981).

α-MSH and its derivatives are known to have potent antipyretic and anti-inflammatory properties, yet they have extremely low toxicity. They can reduce production of host cells' proinflammatory mediators in vitro, and can also reduce production of local and systemic reactions in animal models for inflammation. The “core” α-MSH sequence (4-10) (SEQ. ID. NO. 2), for example, has learning and memory behavioral effects but little antipyretic and anti-inflammatory activity. In contrast, the active message sequence for the antipyretic and anti-inflammatory activities resides in the C-terminal amino-acid sequence of α-MSH, that is, lysine-proline-valine (“Lys-Pro-Val” or “KPV”) (SEQ. ID. NO. 1). This tripeptide has activities in vitro and in vivo that parallel those of the parent molecule. The anti-inflammatory activity of α-MSH and/or its derivatives is disclosed in the following patents are references which are hereby incorporated by reference: U.S. Pat. No. 5,028,592, issued on Jul. 2, 1991 to Lipton, J. M., entitled Antipyretic and Anti-inflammatory Lys Pro Val Compositions and Method of Use; U.S. Pat. No. 5,157,023, issued on Oct. 20, 1992 to Lipton, J. M., entitled Antipreytic and Anti-inflammatory Lys Pro Val Compositions and Method of Use; see also Catania, A., et. al., α-Melanocyte Stimulating Hormone in the Modulation of Host Reactions, Endocr. Rev. 14, 564-576 (1993); Lipton, J. M., et.al., Anti-inflammatory Influence of the Neuroimmunomodulator of α-MSH, Immunol. Today 18, 140-145 (1997); Rajora, N., et. al., α-MSH Production Receptors and Influence on Neopterin, in a Human Monocyte/macrophage Cell Line, J. Leukoc. Biol. 59, 248-253 (1996); Star, R. A., et. al., Evidence of Autocrine Modulation of Macrophage Nitric Oxide Synthase by α-MSH, Proc. Nat'l. Acad. Sci. (USA) 92, 8015-8020 (1995); Lipton, J. M., et.al., Anti-inflammatory Effects of the Neuropeptide α-MSH in Acute Chronic and Systemic inflammation, Ann. N.Y. Acad. Sci. 741, 137-148 (1994); Fajora, N., et. al., α-MSH Modulates Local and Circulating tumor Necrosis Factor α in Experimental Brain Inflammation, J. Neuroosci, 17, 2181-2186 (1995); Richards, D. B., et. al., Effect of a-MSH (11-13) (lysine-proline-valine) on Fever in the Rabbit, Peptides 5, 815-817 (1984); Hiltz, M. E., et. al., Anti-inflammatory Activity of a COOH-terminal Fragment of the Neuropeptide α-MSH, FASEB J. 3, 2282-2284 (1989).

In a preferred embodiment of the invention, these anti-inflammatory activities are most particularly associated with the C-terminal amino-acid sequence—KPV. This tripeptide, along with α-MSH and its derivatives, are effective over a very broad range of concentrations, including picomolar concentrations that normally occur in human plasma.

As discussed in the background section, a topical treatment for sunburn is desired. For treatment of this condition, α-MSH and/or its derivatives can be applied locally to the site of the inflammation by methods known in the art. For example, α-MSH and/or its derivatives can be dissolved in solutions such as phosphate buffer saline, hyalurinate, methylcellulose, carboxymethlcellulose, or ethanol. Common carriers such as cream, ointment, balm, aerosol foam, aerosol spray, pump spray, gel, stick, liquid, or absorbent material can carry α-MSH and/or its derivatives as active ingredients for treating sunburn. These carriers can be applied to the site of the inflammation by spray, absorbent material wipes, swabs, bandages, or fingers.

More specifically, the preferred embodiment of the invention is to dissolve α-MSH and/or its derivatives in a cream-based carrier. The cream containing the solvated α-MSH and/or its derivatives is then spread with fingers on the inflamed skin.

Another preferred embodiment of the invention is to dissolve α-MSH and/or its derivatives in a liquid-based carrier that also contains a skin soothing ingredient and a topical anesthetic. Skin soothing ingredients include, but are not limited to, aloe vera, tocopheryl acetate, menthol, camphor, eucalyptus oil, calamine or any combination thereof. Topical anesthetics include, but are not limited to, lidocaine hydrochloride, benzocaine, pramoxine hydrochloride, or any combination thereof. The carrier containing the solvated α-MSH and/or its derivatives may be stored in a pressurized canister with a spray top. Upon release of the carrier by a release valve or other mechanisms from the pressurized canister, an aerosol spray is formed. The user directs the spray to the inflamed area.

Another preferred embodiment of the invention is a treatment packet with a wipe made of absorbent material that is treated with α-MSH and/or its derivatives that have been dissolved into a liquid-based carrier. The process for making wipes of absorbent material is well known in the art. For example, baby wipes, moist towelettes, make-up removal cloths, and alcohol swabs are all wipes of absorbent material. Commercial examples of such wipes include Chubs® Baby Soft Wipes, Dexus® Antibacterial Hand Wipes, Dexus® Makeup Remover Wipes, Tinactin® Sports Wipes for Athlete's Foot, and B-D® Alcohol Swabs. Treatment of the wipe's absorbent material is accomplished by first soaking the absorbent material in a solution of α-MSH and/or its derivatives. The wipe remains in a liquid-impermeable packaging until use, when the package is opened and the wet wipe is applied to the sunburned skin. The process for making liquid-impermeable packages are well known in the art. For example, packages made of layers of paper, metal foil, and metal foil coated paper are commonly used for packaging wipes of absorbent material. For example, moist towelettes, such as Massengill® Feminine Cleansing Soft Cloth Towelettes, and alcohol swabs, such as B-D® Alcohol Swabs, are packaged in this manner.

Another preferred embodiment is a method of preventing sunburn by applying α-MSH and/or its derivatives that are carried by a cream, ointment, balm, aerosol foam, aerosol spray, pump spray, gel, stick, liquid, or absorbent material to skin exposed to the sun.

The following examples demonstrate the application of α-MSH and its derivatives to reduce sunburn inflammation.

One of skill in the art will be able to determine the appropriate dosage required for varying degrees of sunburn.

### EXAMPLE I

This example illustrates the biological functional equivalents of α-MSH and/or its derivatives.

Although specific amino acid sequences described here are effective, it is clear to those familiar with the art that amino acids can be substituted or deleted without altering the effectiveness of the peptides. Further, it is known that stabilization of the α-MSH sequence can greatly increase the activity of the peptide and that substitution of D-amino acid forms for L-forms can improve or decrease the effectiveness of the peptides. For example, a stable analog of α-MSH, [Nle⁴,D-Phe⁷]α-MSH, which is known to have marked biological activity on melanocytes and melanoma cells, is approximately ten times more potent than the parent peptide in reducing fever. Further, adding amino acids to the C-terminal of α-MSH(11-13) (SEQ. ID. NO. 1) sequence can reduce or enhance antipyretic potency. Addition of glycine to form the 10-13 sequence (SEQ. ID. NO. 5) slightly decreased potency; the 9-13 sequence (SEQ. ID. NO. 6) was almost devoid of activity, whereas the potency of the 8-13 sequence (SEQ. ID. NO. 7) was greater than that of the 11-13 sequence (SEQ. ID. NO. 1). It is known that Ac-[D-K11]-α-MSH 11-13-NH2 has the same general potency as the L-form of the tripeptide α-MSH (11-13) (SEQ. ID. NO. 1). However, substitution with D-proline in position 12 of the tripeptide rendered it inactive. see e.g. Holdeman, M., et. al., Antipyretic Activity of a Potent α-MSH Analog, Peptides 6, 273-5 (1985). Deeter, L. B., et. al., Antipyretic Properties of Centrally Administered α-MSH Fragments in the Rabbit, Peptides 9, 1285-8 (1989). Hiltz, M. E., Anti-inflammatory Activity of α-MSH (11-13) Analogs: Influences of Alterations in Stereochemistry, Peptides 12, 767-71 (1991).

Biological functional equivalents can also be obtained by substitution of amino acids having similar hydropathic values. Thus, for example, isoleucine and leucine, which have a hydropathic index +4.5 and +3.8, respectively, can be substituted for valine, which has a hydropathic index of +4.2, and still obtain a protein having like biological activity. Alternatively, at the other end of the scale, lysine (−3.9) can be substituted for arginine (−4.5), and so on. In general, it is believed that amino acids can be successfully substituted where such amino acid has a hydropathic score of within about ±1 hydropathic index unit of the replaced amino acid.

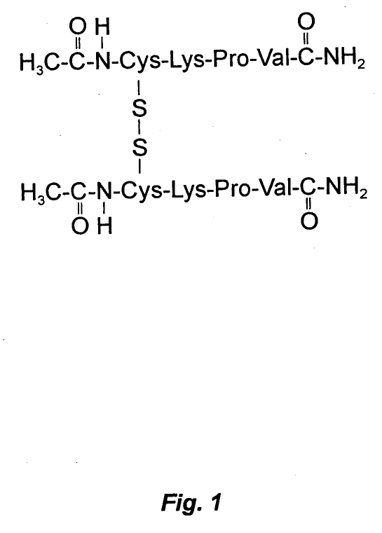

Furthermore, these modified analogs of α-MSH and/or its derivatives can also form dimers as exemplified by the KPV dimer in FIG. 1.

### EXAMPLE II

A person spends time in the sun with exposed skin. Within hours, red and painful inflammation appears on the exposed area. An examination by a physician determines that the patient has sunburn from over-exposure to ultraviolet radiation. Treatment can include topical application of pharmacologically effective amounts of α-MSH and/or its derivatives carried in a cream, an ointment, a balm, an aerosol foam, an aerosol spray, a pump spray, a gel, a stick, a liquid, or the absorbent material of a wipe. The topical treatment can be applied once or multiple times according to the discretion of the physician until the inflammation is resolved.

### EXAMPLE III

A person plans on spending time in the sun with exposed skin and desires to prevent sunburn. Prior to exposure, the person topically applies a pharmacologically effective amount of α-MSH and/or its derivatives carried in a cream, an ointment, a balm, an aerosol foam, an aerosol spray, a pump spray, a gel, a stick, a liquid, or the absorbent material of a wipe. The topical treatment can be re-applied once or multiple times as necessary to prevent sunburn, especially after towel drying, swimming, or perspiring.

### EXAMPLE IV

Some sample formulations include combining α-MSH and/or its derivatives in a:
a. Cream formulation also containing water, glycerin, mineral oil, lanolin, stearic acid, cetyl alcohol, aloe vera, coconut oil, tocopheryl acetate, fragrance, and coloring;
b. Cream formulation also containing water, stearic acid, camphor, menthol, and eucalyptus oil;
c. Ointment also containing aloe vera, petrolatum, and mineral oil;
d. Pump Spray also containing lidocaine HCl, water, aloe vera, glycerin, fragrance, and coloring;
e. Aerosol Spray also containing lidocaine HCl, aloe vera, isobutane, and cetyl acetate;
f. Gel formulation also containing lidocaine HCl, water, aloe vera, glycerin, fragrance, and coloring; and a
g. Foam formulation also containing water, isobutane, stearic acid, aloe vera, tocopherol acetate, fragrance and coloring.
   It will be understood by one skilled in the art that other inactive ingredients can be combined with the above formulations.

The preceding examples demonstrate the application of α-MSH and/or its derivatives to reduce and prevent sunburn inflammation. These are only examples and are not intended to limit the invention to these examples. It is understood that modifying the examples above does not depart from the spirit of the invention. It is further understood that the examples can be applied on its own or in combination with each other.

1. A sunburn treatment comprising:
a carrier to be applied to cutaneous inflammation caused by exposure to ultraviolet radiation;
at least one polypeptide including an amino acid sequence selected from a group consisting of KPV (SEQ. ID. NO. 1), MEHFRWG (SEQ. ID. NO. 2), HFRWGKPV (SEQ. ID. NO. 3), SYSMEHFRWGKPV (SEQ. ID. NO. 4), or a biologically functional equivalent of any of the foregoing;
wherein the at least one polypeptide is at a level to effectively treat the cutaneous inflammation; and
wherein the carrier carries the at least one polypeptide.

2-67. Canceled.


SEQUENCE LISTING (7 sequences)

SEQ ID NO: 1 - protein, 3 aa, Artificial Sequence
  Designed polypeptide with anti-inflammatory, anti-bacterial, anti-fungal and antipyretic properties.
KPV

SEQ ID NO: 2 - protein, 10 aa, Artificial Sequence
  Designed polypeptide with anti-inflammatory, anti-bacterial, anti-fungal and antipyretic properties.
MEHFRWGKPV

SEQ ID NO: 3 - protein, 8 aa, Artificial Sequence
  Designed polypeptide with anti-inflammatory, anti-bacterial, anti-fungal and antipyretic properties.
HFRWGKPV

SEQ ID NO: 4 - protein, 13 aa, Artificial Sequence
  alpha-MSH with anti-inflammatory, anti-bacterial, anti-fungal and antipyretic properties.
SYSMEHFRWGKPV

SEQ ID NO: 5 - protein, 4 aa, Artificial Sequence
  Designed polypeptide with anti-inflammatory, anti-bacterial, anti-fungal and antipyretic properties.
GKPV

SEQ ID NO: 6 - protein, 5 aa, Artificial Sequence
  C-terminal sequences of alpha-MSH
WGKPV

SEQ ID NO: 7 - protein, 6 aa, Artificial Sequence
  Designed polypeptide with anti-inflammatory, anti-bacterial, anti-fungal and antipyretic properties.
RWGKPV